SEQUENCE LISTING (16 sequences)

SEQ ID NO: 1 - DNA, 17 bp, Artificial Sequence
  primer
gcgagtccgaggatggc

SEQ ID NO: 2 - DNA, 20 bp, Artificial Sequence
  primer
atccgcaggttctctcggta

SEQ ID NO: 3 - DNA, 15 bp, Artificial Sequence
  probe
gagacacggaacatg

SEQ ID NO: 4 - DNA, 15 bp, Artificial Sequence
  probe
gagacacggaacatg

SEQ ID NO: 5 - DNA, 17 bp, Artificial Sequence
  primer
gccgcgagtccgagaga

SEQ ID NO: 6 - DNA, 20 bp, Artificial Sequence
  primer
atccgcaggttctctcggta

SEQ ID NO: 7 - DNA, 15 bp, Artificial Sequence
  probe
ccggagtattgggac

SEQ ID NO: 8 - DNA, 15 bp, Artificial Sequence
  probe
ccggaatattgggac

SEQ ID NO: 9 - DNA, 14 bp, Artificial Sequence
  primer
atggagccgcgggc

SEQ ID NO: 10 - DNA, 23 bp, Artificial Sequence
  primer
gtccactcggtcaatctgtgagt

SEQ ID NO: 11 - DNA, 13 bp, Artificial Sequence
  probe
ggggccggagtat

SEQ ID NO: 12 - DNA, 15 bp, Artificial Sequence
  probe
gagaggcctgagtat

SEQ ID NO: 13 - DNA, 25 bp, Artificial Sequence
  primer
acaccatccagataatgtatggctg

SEQ ID NO: 14 - DNA, 19 bp, Artificial Sequence
  primer
aagagcgcaggtcctcgtt

SEQ ID NO: 15 - DNA, 16 bp, Artificial Sequence
  probe
gggtaccaccagtacg

SEQ ID NO: 16 - DNA, 16 bp, Artificial Sequence
  probe
gggtatgaacagcacg

(19) United States
(12) Patent Application Publication
(10) Pub. No.: US 2022/0177968 A1
(43) Pub. Date: Jun. 9, 2022

(54) METHODS FOR DETECTING OR REDUCING THE INCIDENCE OF ADVERSE DRUG REACTIONS

(71) Applicants: Jean-Claude ROUJEAU (deceased), Chatenay (FR); CHANG GUNG MEMORIAL HOSPITAL, LINKOU, Taoyuan City (TW); UNIVERSITY OF FREIBURG, Freiburg (DE)

(72) Inventors: Jean-Claude ROUJEAU (deceased), Chatenay (FR); Wen-Hung CHUNG, Taoyuan City (TW); Maja MOCKENHAUPT, Freiburg (DE)

(21) Appl. No.: 17/438,239

(22) PCT Filed: Mar. 12, 2020
(86) PCT No.: PCT/CN2020/078994

§ 371 Date: Sep. 10, 2021

(30) Foreign Application Priority Data
Mar. 12, 2019 (AU) 2019900816

(51) Int. Cl. C12Q 1/6883

(52) U.S. Cl. CPC C12Q 1/6883; C12Q 2600/172; C12Q 2600/156; C12Q 2600/106

(57) ABSTRACT

Methods for assessing the risk of developing adverse drug reaction in a subject in need of such assessment are provided, comprising the step of detecting the presence of an HLA-B allele in a sample obtained from the subject, wherein the presence of allele is indicative of the subject having an increased risk of developing the adverse drug reaction. Also provided are methods of treating or reducing the incidence of the adverse drug reaction.

### CROSS-REFERENCE TO RELATED APPLICATION

This application claims priority to Australian Application No. 2019900816, filed on 12 Mar. 2019, the entire disclosure of which is incorporated herein by reference.

### FIELD OF THE INVENTION

The present invention relates to methods for predicting the risk of developing adverse drug reactions in a subject, and more particularly to assess the risk of developing severe cutaneous adverse drug reactions in response to carbamazepine. The present invention is also related to methods for reducing the incidence of or treating the adverse drug reaction in response to carbamazepine.

### BACKGROUND OF THE INVENTION

Carbamazepine (CBZ) is widely used as the first generation antiepileptic drug for the treatment of neurological diseases, such as epilepsy, bipolar disorder, and trigeminal neuralgia. Although effective for treating neurological diseases, CBZ may cause adverse drug reactions ranging from mild maculopapular exanthema (MPE) to life-threatening severe cutaneous adverse reactions (SCAR), including Stevens-Johnson syndrome (SJS), toxic epidermal necrolysis (TEN), and drug reaction with eosinophilia and systemic symptoms (DRESS).

SJS/TEN is characterized as epidermal detachment with high mortality rate ranging from 10 to 50%. HLA-B*15:02 was found to be strongly associated with CBZ-SJS/TEN in Han Chinese and populations of Asian ancestry, such as Thailand, Malaysia, Singapore, Hong Kong, Vietnam and India. Genetic screening of HLA-B*15:02 prior to the use of CBZ for patients with Asian ancestry is recommended by health authorities in many countries, such as USFDA and Taiwan FDA. In addition, HLA-A*31:01 is associated with CBZ hypersensitivity reactions in Europeans (McCormack M et al., N Engl J Med 2011; 364(12):1134-1143). Recent study revealed that HLA-A*31:01 is more related to CBZ-MPE/DRESS comparing to CBZ-SJS/TEN in European population (Genin E et al., Pharmacogenomics J 2014; 14(3):281-288).

There is an unmet need for the identification of biomarkers, particularly measurable in vivo and less invasive, to accurately assess the risk of a subject in developing an adverse drug reaction, especially CBZ induced SCAR, in an individual. The present invention satisfies this and other needs.

### SUMMARY OF THE INVENTION

The present invention provides a method for accessing the risk for developing an adverse drug reaction in a subject, comprising the steps of (a) detecting the presence of an HLA-B*57:01 allele, an HLA-B*38:01 allele or the combination thereof in the sample of the subject, and (b) identifying the subject as having an increased risk of developing the adverse drug reaction if the HLA-B*57:01 allele, the HLA-B*38:01 allele or the combination thereof is present.

Also provided is a method for assessing the risk of developing an adverse drug reaction and treating the adverse drug reaction, comprising the steps of (a) detecting the presence of an HLA-B*57:01 allele, an HLA-B*38:01 allele or the combination thereof in the sample of the subject, (b) identifying the subject as having an increased risk of developing the cutaneous adverse drug reaction if the HLA-B*57:01 allele, the HLA-B*38:01 allele or the combination thereof is present and (c) administering a medication to treat the adverse drug reaction.

The present invention is also directed to a method for assessing the risk of developing an adverse drug reaction and reducing the incidence of the adverse drug reaction, comprising the steps of (a) detecting the presence of an HLA-B*57:01 allele, an HLA-B*38:01 allele or the combination thereof in the sample of the subject, (b) identifying the subject as having an increased risk of developing the cutaneous adverse drug reaction if the HLA-B*57:01 allele, the HLA-B*38:01 allele or the combination thereof is present and (c) administering a treatment that is not an anticonvulsant.

Also provided is the use of an agent for detecting the HLA-B*57:01 allele, an HLA-B*38:01 allele or the combination thereof in the manufacture of a diagnostic kit to evaluate the risk of developing an adverse drug reaction induced by an anti-convulsant.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The invention will become more apparent when read with the detailed description which follow.

### DETAILED DESCRIPTION OF THE INVENTION

As used herein, the articles “a” and “an” refer to one or more than one (i.e., at least one) of the grammatical object of the article.

The use of the term “or” in the claims is used to mean “and/or” unless explicitly indicated to refer to alternatives only or the alternatives are mutually exclusive, although the disclosure supports a definition that refers to only alternatives and “and/or.”

“Patient” or “subject” as used herein refers to a mammalian subject in need of an anticonvulsant medication now or in the future.

The terms “Caucasian” and “European Descent” are used interchangeably, referring to a race of humankind native to Europe, North Africa, and southwest Asia and classified according to a specific physical feature, mainly light skin pigmentation.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

The present invention provides a method for accessing the risk of developing an adverse drug reactions in a subject, comprising the steps of (a) detecting the presence of an HLA-B*57:01 allele in the sample of the subject, and (b) identifying the subject as having an increased risk of developing the adverse drug reaction if the HLA-B*57:01 allele is present. In an exemplary embodiment, the adverse drug reaction is severe cutaneous adverse reactions (SCAR) selected from SJS or TEN. In another exemplary embodiment, the adverse drug reaction is SCAR, including SJS, TEN or DRESS.

In some embodiments, the combinations of HLA-B*57:01/HLA-B*38:01, HLA-B*57:01/HLA-A*31:01 or HLA-B*57:01/HLA-B*38:01/HLA-A*31:01 synergistically predict the risk of developing an adverse drug reaction, wherein the adverse drug reaction is SCAR, including SJS, TEN or DRESS. The adverse drug reaction can be induced by an anticonvulsant, including by not limited to carbamazepine, oxcarbazepine, phenytoin, fosphenytoin, phenobarbital, and lamotrigine. In an exemplary embodiment, the anticonvulsant is an aromatic anticonvulsant comprises an aromatic ring.

In certain embodiments, the risk alleles (HLA-B*57:01, HLA-B*38:01 or HLA-A*31:01) can be detected by any method known in the art, including but not limited to, HLA typing, serological or microcytotoxicity methods, or the detection of an equivalent genetic marker of the allele. An “equivalent genetic marker” of the risk allele refers to a genetic marker that is linked to the allele of interest (it displays a linkage disequilibrium with the allele of interest) and can be, for example, an SNP (single nucleotide polymorphism), a microsatellite marker or other kinds of genetic polymorphisms. In one embodiment, the genomic DNA is hybridized to a probe that is specific for the variant of interest. The probe may be labeled for direct detection, or contacted by a second, detectable molecule that specifically binds to the probe. Alternatively, cDNA, RNA, or protein product of the variant can be detected.

In some embodiments, the HLA-B*57:01 allele is detected by contacting the sample of the subject with a forward primer with a nucleotide sequence at least 90%, 95% or 100% identical to SEQ ID NO:1 (GCGAGTCCGAGGATGGC) a reverse primer with a nucleotide sequence at least 90%, 95% or 100% identical to SEQ ID NO: 2 (ATCCGCAGGTTCTCTCGGTA); Probe 1 with a nucleotide sequence at least 90%, 95% or 100% identical to SEQ ID NO: 3 (GAGACACGGAACATG) and/or Probe 2 with a nucleotide sequence at least 90%, 95% or 100% identical to SEQ ID NO:4 (GAGACACGGAACATG). In other embodiments, the HLA-B*38:01 allele is detected by contacting the sample of the subject with a forward primer with a nucleotide sequence at least 90%, 95% or 100% identical to SEQ ID NO: 5 (GCCGCGAGTCCGAGAGA); a reverse primer with a nucleotide sequence at least 90%, 95% or 100% identical to SEQ ID NO: 6 (ATCCGCAGGTTCTCTCGGTA); Probe 1 with a nucleotide sequence at least 90%, 95% or 100% identical to SEQ ID NO: 7(CCGGAGTATTGGGAC) and/or Probe 2 with a nucleotide sequence at least 90%, 95% or 100% identical to SEQ ID NO: 8 (CCGGAATATTGGGAC). In other embodiments, the HLA-A*31:01 allele is detected by contacting the sample of the subject with a forward primer 1 with anucleotide sequence at least 90%, 95% or 100% identical to SEQ ID No.9 (ATGGAGCCGCGGGC); a reverse primer with a nucleotide sequence at least 90%, 95% or 100% identical to SEQ ID NO:10 (GTCCACTCGGTCAATCTGTGAGT); Probe 1: with a nucleotide sequence at least 90%, 95% or 100% identical to SEQ ID NO: 11 (GGGGCCGGAGTAT); Probe 2 with a nucleotide sequence at least 90%, 95% or 100% identical to SEQ ID NO:12 (GAGAGGCCTGAGTAT); a forward primer with a nucleotide sequence at least 90%, 95% or 100% identical to SEQ ID NO: 13 (ACACCATCCAGATAATGTATGGCTG), a reverse primer with a nucleotide sequence at least 90%, 95% or 100% identical to SEQ ID NO: 14 (AAGAGCGCAGGTCCTCGTT); Probe 1: with a nucleotide sequence at least 90%, 95% or 100% identical to SEQ ID 15 (GGGTACCACCAGTACG) and/or Probe 4 with a nucleotide sequence at least 90%, 95% or 100% identical to SEQ ID NO: 16 (GGGTATGAACAGCACG). The HLA allele of interest is detected by the specific binding between the HLA allele and the primer and/or probe.

The risk HLA alleles can be detected by direct detection of regions/nucleotides within the allele using genomic DNAs prepared from the sample of the subject, including but not limited to blood, saliva, urine or hair.

Another aspect of the present invention provides a method of pharmacogenomics profiling comprising the step of determining the presence of HLA-B*57:01 in the sample of a subject. In one embodiment, the presence of HLA-B*38:01 and/or HLA-A*31:01 is also determined. In another embodiment, the pharmacogenomics profiling comprises the determination of other risk factors associated with the predisposition for any disease or other medical condition, including adverse drug reactions. Further provided is a method of screening and or identifying medications that can be used to treat adverse drug reactions by using HLA-B*57:01 allele, an HLA-B*38:01 allele, the combinations of HLA-B*57:01/HLA-B*38:01 or HLA-B*57:01/HLA-A*31:01 as a target in drug development.

The present invention is also directed to methods for assessing the risk of developing an adverse drug reaction and reducing the incidence of the adverse drug reaction, comprising the steps of (a) detecting the presence of an HLA-B*57:01 allele in the sample of the subject, (b) identifying the subject as having an increased risk of developing the cutaneous adverse drug reaction if the HLA-B*57:01 allele is present and (c) administering a treatment that is not an anticonvulsant.

In an exemplary embodiment, the method of reducing the incidence of an adverse drug reaction is by administering a treatment that is not carbamazepine. In another exemplary embodiment, the method of treating an adverse drug reaction is by administering a medication to treat the adverse drug reaction including but not limited to fluid, corticosteroid, intravenous immunoglobulin, cyclosporine, anti-TNF-α agent or plasmapheresis.

The use of an agent for detecting the HLA-B*57:01 allele, an HLA-B*38:01 allele or the combination thereof in the manufacture of a diagnostic kit to evaluate the risk of developing an adverse drug reaction induced by an anti-convulsant is provided. In an exemplary embodiment, the diagnostic kit further comprising an agent for detecting the HLA-A*31:01, in combination with (a) an agent for detecting the HLA-B*57:01 allele or (b) an agent for detecting the HLA-B*57:01 allele and an agent for detecting the HLA-B*38:01 allele.

Embodiments of the present invention are illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the invention. During the studies described in the following examples, conventional procedures were followed, unless otherwise stated. Some of the procedures are described below for illustrative purpose.

### Description of Materials and Methods Used in the Examples

A total of 28 patients of European descent with CBZ-SJS/TEN were enrolled in this study. SJS/TEN is characterized by a rapidly developing exanthema of purpuric macules and target-like lesions with skin detachment accompanied by hemorrhagic-erosive mucosal involvement. Skin detachment in SJS patients affects less than 10% of body surface area (BSA), while TEN patients have skin detachment greater than 30% of BSA. Detachment of 10-30% is defined as SJS/TEN-overlap, reflecting a continuum of severity variants of one disease entity. Patients with CBZ-DRESS were also enrolled for comparison.

HLA-A and HLA-B genotypes of patients with CBZ-SJS/TEN were determined by SeCore HLA sequence-based typing (Invitrogen, Life Technologies, USA). Furthermore, the HLA genotypes for patients with CBZ-DRESS and general population (the control group) were also determined.

Statistical analyses were performed using SPSS 21.0 (IBM, Armonk, N.Y.). Comparisons of genotype frequencies between groups were performed using Fisher exact tests. Differences were considered statistically significant at p-values of less than 0.05.

Table 1 shows HLA-B*57:01 was strongly associated with CBZ-SJS/TEN compared to European general population controls (Table 1). HLA-B*57:01 allele was observed in 39.29% (11/28) of European descent patients with CBZ-SJS/TEN, but only in 6.69% (593/8862) of European general population controls (OR=9.0, 95% CI=4.2-19.4; P=9.62×10⁻⁷). HLA-B*38:01 was also associated with CBZ-SJS/TEN (OR=4.3, 95% CI=1.5-12.5; P=0.02) (Table 1). The results indicated that the presence of HLA-B*57:01 or HLA-B*38:01 is associated with increased risk of CBZ-SJS/TEN.

TABLE 1
Association of HLA-A/B allele and European descent
patients with CBZ-induced SJS/TEN.

| HLA | SJS/TEN | General population* | | P |
|---|---|---|---|---|
| Genotype | N (%) | N (%) | OR (95% CI) | values |
| HLA-A | | | | |
| A*01:01 | 14/28 (50.0%) | 2731/8862 (30.82%) | 2.2 (1.1-4.7) | 0.039 |
| A*02:01 | 11/28 (39.29%) | 5118/8862 (57.75%) | 0.5 (0.2-1.0) | 0.056 |
| A*24:02 | 3/28 (10.71%) | 1572/8862 (17.74%) | 0.6 (0.2-1.8) | 0.460 |
| A*26:01 | 5/28 (17.86%) | 530/8862 (5.99%) | 3.5 (1.3-9.0) | 0.024 |
| A*31:01 | 4/28 (14.29%) | 396/8862 (4.52%) | 3.5 (1.2-10.2) | 0.035 |
| HLA-B | | | | |
| B*08:01 | 8/28 (28.57%) | 1835/8862 (20.71%) | 1.5 (0.7-3.5) | 0.348 |
| B*13:02 | 3/28 (10.71%) | 549/8862 (6.2%) | 1.8 (0.5-6.0) | 0.251 |
| B*15:02 | 0/28 (0%) | 3/8862 (0.034%) | 44.4 (2.2-879.5) | 1 |
| B*18:01 | 3/28 (10.71%) | 838/8862 (9.46%) | 1.1 (0.3-3.8) | 0.744 |
| B*38:01 | 4/28 (14.29%) | 330/8862 (3.72%) | 4.3 (1.5-12.5) | 0.020 |
| B*44:02 | 1/28 (3.57%) | 1428/8862 (16.11%) | 0.2 (0.0-1.4) | 0.074 |
| B*44:03 | 4/28 (14.29%) | 726/8862 (8.19%) | 1.9 (0.6-5.4) | 0.284 |
| B*57:01 | 11/28 (39.29%) | 593/8862 (6.69%) | 9.0 (4.2-19.4) | 9.62 × |
| | | | | 10⁻⁷ |

The association of HLA-B*57:01, HLA-A*31:01, and HLA-B*38:01 and CBZ-SCAR was evaluated by combining the data of the patients of European ancestry with CBZ-SCAR. Combined HLA-B*57:01/HLA-A*31:01/HLA-B*38:01 was present in 63.16% (24/38) of CBZ-SCAR patients, and only in 14.23% (1261/8862) of European general population (OR=10.3, 95% CI=5.3-20.0; P=6.96×10⁻¹²) (Table 2). The results indicated that the presence of HLA-B*57:01/HLA-A*31:01/HLA-B*38:01 is associated with increased risk of CBZ-SJS, TEN and DRESS in patients of European descent.

TABLE 2
Association of HLA-B*57:01, HLA-A*31:01, HLA-B*38:01 and CBZ-induced SCAR
in patients with European descent.

| | | | General | | | |
|---|---|---|---|---|---|---|
| | SCAR case | | population | Odds ratio | | Sensitivity |
| Subgroup | N (%) | | N (%) | (95% CI) | P value | (%) |
| HLA-B*57:01 | | | | | | |
| CBZ-SJS/TEN | 11/28 | (39.29%) | 593/8862 (6.69%) | 9.0 (4.2-19.4) | 9.62 × 10⁻⁷ | 39.29 |
| CBZ-DRESS* | 0/10 | (0%) | 593/8862 (6.69%) | 0.7 (0.0-11.3) | 1 | 0 |
| HLA-A*31:01 | | | | | | |
| CBZ-SJS/TEN | 4/28 | (14.29%) | 396/8862 (4.52%) | 3.6 (1.2-10.3) | 0.035 | 14.29 |
| CBZ-DRESS* | 7/10 | (70%) | 396/8862 (4.52%) | 49.9 (12.9-193.6) | 4.0 × 10⁻⁸ | 70.00 |
| HLA-B*38:01 | | | | | | |
| CBZ-SJS/TEN | 4/28 | (14.29%) | 330/8862 (3.72%) | 4.3 (1.5-12.5) | 0.020 | 14.29% |
| CBZ-DRESS* | 0/10 | (0%) | 330/8862 (3.72%) | 1.2 (0.1-21.0) | 1 | 0 |
| HLA-B*57:01/A*31:01/B*38:01 | | | | | | |
| SCAR | 24/38 | (63.16%) | 1261/8862 (14.23%) | 10.3 (5.3-20.0) | 6.96 × 10⁻¹² | 63.16 |
| HLA-B*57:01 | | | | | | |
| CBZ-SJS/TEN | 11/28 | (39.29%) | 593/8862 (6.69%) | 9.0 (4.2-19.4) | 9.62 × 10⁻⁷ | 39.29 |
| CBZ-DRESS | 0/10 | (0%) | 593/8862 (6.69%) | 0.7 (0.0-11.3) | 1 | 0 |
| CBZ-SCAR | 11/38 | (28.95%) | 593/8862 (6.69%) | 5.7 (2.8-11.5) | 2.85 × 10⁻⁵ | 28.95 |
| HLA-A*31:01 | | | | | | |
| CBZ-SJS/TEN | 4/28 | (14.29%) | 396/8862 (4.52%) | 3.6 (1.2-10.3) | 0.035 | 14.29 |
| CBZ-DRESS | 7/10 | (70.00%) | 396/8862 (4.52%) | 49.9 (12.9-193.6) | 4.04 × 10⁻⁸ | 70.00 |
| CBZ-SCAR | 11/38 | (28.95) | 396/8862 (4.52%) | 8.7 (4.3-17.7) | 6.29 × 10⁻⁷ | 28.95 |
| HLA-B*38:01 | | | | | | |
| CBZ-SJS/TEN | 4/28 | (14.29%) | 330/8862 (3.72%) | 4.3 (1.5-12.5) | 0.020 | 14.29 |
| CBZ-DRESS | 0/10 | (0%) | 330/8862 (3.72%) | 1.2 (0.1-21.0) | 1 | 0 |
| CBZ-SCAR | 4/38 | (10.53%) | 330/8862 (3.72%) | 3.0 (1.1-8.6) | 0.053 | 10.53 |
| HLA-B*57:01/A*31:01 | | | | | | |
| CBZ-SJS/TEN | 14/28 | (50.00%) | 989/8862 (11.16%) | 8.0 (3.8-16.7) | 4.33 × 10⁻⁷ | 50.00 |
| CBZ-DRESS | 7/10 | (70.00%) | 989/8862 (11.16%) | 18.6 (4.8-71.9) | 1.95 × 10⁻⁵ | 70.00 |
| CBZ-SCAR | 21/38 | (55.26%) | 989/8862 (11.16%) | 9.8 (5.2-18.7) | 5.05 × 10⁻¹¹ | 55.26 |
| HLA-B*57:01/B*38:01 | | | | | | |
| CBZ-SJS/TEN | 14/28 | (50.00%) | 923/8862 (10.42%) | 8.6 (4.1-18.1) | 1.85 × 10⁻⁷ | 50.00 |
| CBZ-DRESS | 0/10 | (0%) | 923/8862 (10.42%) | 0.4 (0-7.0) | 0.613 | 0 |
| CBZ-SCAR | 14/38 | (36.84) | 923/8862 (10.42%) | 5.0 (2.6-9.7) | 1.60 × 10⁻⁵ | 36.84 |
| HLA-B*57:01/A*31:01/B*38:01 | | | | | | |
| CBZ-SJS/TEN | 17/28 | (60.71%) | 1261/8862 (14.23%) | 9.3 (4.4-19.9) | 1.93 × 10⁻⁸ | 60.71 |
| CBZ-DRESS | 7/10 | (70.00%) | 1261/8862 (14.23%) | 14.1 (3.6-54.5) | 9.68 × 10⁻⁵ | 70.00 |
| CBZ-SCAR | 24/38 | (63.16%) | 1261/8862 (14.23%) | 10.3 (5.3-20.0) | 6.96 × 10⁻¹² | 63.16 |

What is claimed is:

1. A method of assessing a risk for developing an adverse drug reaction in a subject, comprising the steps of
(a) detecting the presence of HLA-B*57:01 allele, HLA-B*38:01 allele or the combination thereof in the sample of the subject, and
(b) identifying the subject as having an increased risk of developing the adverse drug reaction if the HLA-B*57:01 allele, HLA-B*38:01 allele or the combination thereof is present.

2. The method of claim 1, wherein the adverse drug reaction is induced by an anti-convulsant.

3. The method of claim 2, wherein the anti-convulsant is carbamazepine.

4. The method of claim 1, wherein the presence of the HLA-B*57:01 or HLA-B*38:01 allele is determined by using oligonucleotides that specifically hybridizes to the allele.

5. The method of claim 4, wherein the oligonucleotide has a nucleotide sequence at least 90% identical to SEQ ID NO:1, SEQ ID NO: 2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16.

6. The method of claim 1, wherein the sample is DNA, RNA, protein, cells, serum, peripheral blood, saliva, urine, hair or skin.

7. The method of claim 1, wherein the subject is an European descent or a Caucasian.

8. The method of claim 1, further comprising detecting the presence of an HLA-A*31:01 allele.

9. The method of claim 1, wherein the adverse drug reaction is Stevens-Johnson syndrome, toxic epidermal necrolysis or drug reactions with eosinophilia and systemic symptoms.

10. A method for assessing the risk of developing an adverse drug reaction and treating the adverse drug reaction, comprising the steps of
(a) detecting the presence of an HLA-B*57:01 allele, an HLA-B*38:01 allele or the combination thereof in the sample of the subject;
(b) identifying the subject as having an increased risk of developing the cutaneous adverse drug reaction if the HLA-B*57:01 allele, HLA-B*38:01 allele or the combination thereof is present; and
(c) administering a medication to treat the adverse drug reaction.

11. A method for assessing the risk of developing an adverse drug reaction and reducing the incidence of the adverse drug reaction, comprising the steps of:
(a) detecting the presence of an HLA-B*57:01 allele, an HLA-B*38:01 allele or the combination thereof in the sample of the subject;
(b) identifying the subject as having an increased risk of developing the cutaneous adverse drug reaction if the HLA-B*57:01 allele, the HLA-B*38:01 allele or the combination thereof is present; and
(c) administering a treatment that is not an anticonvulsant.

12. The use of an agent for detecting the HLA-B*57:01 allele, an HLA-B*38:01 allele or the combination thereof in the manufacture of a diagnostic kit to evaluate the risk of developing an adverse drug reaction induced by an anti-convulsant.

13. The use according to claim 12, wherein the agent comprises an oligonucleotide specifically hybridizes to the HLA-B*57:01 allele or an HLA-B*38:01 allele.

14. The use of claim 13, wherein the oligonucleotide has a nucleotide sequence at least 90% identical to SEQ ID NO:1, SEQ ID NO: 2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16.

15. The use according to claim 12, wherein the adverse drug reaction is Stevens-Johnson syndrome, toxic epidermal necrolysis or drug reactions with eosinophilia and systemic symptoms.

16. The use according to claim 12, wherein the anti-convulsant is carbamazepine.

17. The use according to claim 12, wherein the diagnostic kit further comprising an agent for detecting the HLA-A*31:01.
